(19) United States
(12) Patent Application Publication
(10) Pub. No.: US 2016/0120853 A1
(43) Pub. Date: May 5, 2016

(54) TACROLIMUS AND ANALOGUES THEREOF FOR MEDICAL USE

(71) Applicant: CHRONOS THERAPEUTICS LIMITED, Oxford (GB)

(72) Inventors: Alexandre AKOULITCHEV, Ewelme, Oxfordshire (GB); Elizabeth MILWAY, Oxford, Oxfordshire (GB); Elizabeth Jane MELLOR, Kennington, Oxford, Oxfordshire (GB); Michael YOUDELL, Oxford, Oxfordshire (GB)

(21) Appl. No.: 14/893,258

(22) PCT Filed: May 22, 2014
(86) PCT No.: PCT/GB2014/051571

§ 371 Date: Nov. 23, 2015

(30) Foreign Application Priority Data
May 24, 2013 (GB) 1309376.0

(51) Int. Cl. A61K 31/436; A61K 9/00

(52) U.S. Cl. CPC A61K 31/436; A61K 9/0053

(57) ABSTRACT

The present invention relates to a method of treating adverse fat accumulation and related conditions with very low doses of tacrolimus or a close structural analogue thereof. In particular this invention relates to treating adverse fat accumulation and/or sarcopenia and/or metabolic syndrome inpatients by administering tacrolimus in very low dose. Most particularly this invention relates to the use of tacrolimus in treating age related fat accumulation.

The present invention relates to a method of treating adverse fat accumulation and related conditions with very low doses of tacrolimus or a close structural analogue thereof. In particular this invention relates to treating adverse fat accumulation and/or sarcopenia and/or metabolic syndrome in patients by administering tacrolimus in very low dose. Most particularly this invention relates to the use of tacrolimus in treating age related fat accumulation.

Tacrolimus (also called fujimycin or FK506) is clinically employed as an immunosuppressant, for example, in patients who have had organ transplants, and for the treatment of ulcerative colitis or certain skin conditions. Tacrolimus is available under trade names such as Prograf®, Advagraf® and Protopic®. Commercially available dosage forms of tacrolimus include capsules containing 0.5 mg, 1 mg, 3 mg and 5 mg and ointments for skin conditions where the concentration is 0.05% to 0.19%. Tacrolimus is most commonly administered twice a day for immunosuppression to prevent rejection of transplanted tissues. The clinically employed dose is generally adjusted to produce a whole blood trough concentration of at least 4 mg/mL when seeking to prevent rejection. This is achieved by employing a recommended initial oral dose (two divided doses every 12 hours) which is in the range 0.075 mg/kg/day to 0.2 mg/kg/day which for an average 70 kg patient required two daily doses of about 2.5 mg to 14 mg. Tacrolimus has also been employed for the treatment of arthritis generally at 3 mg per day. Possibly the lowest dose employed for immunosuppression was recorded was for the treatment of myasthenia gravis was 2-3 mg per day (Kanshi et al., J. Neurol. Neurosurg. Psychiatry 2005; 76: 448-450) but this was together with up to 50 mgs per day of prednisolone (and was administered to a lady who may have had low body weight).

It is believed that these established clinical uses for tacrolimus operate via a mechanism which acts via calmodulin to activate calcineurin which thus inhibits both T-lymphocyte signal transduction and IL-2 transcription. These are dose dependent mechanism so that greater the amount of tacrolimus administered the greater the immunosuppression. This mechanism is not involved in the present invention which is fortunate because immunosuppression is desirable when treating the conditions set forth above, but since it leads to a reduction in immunovigilance, it is not without its disadvantages such as increased risk of infection or lymphoma.

WO 2011/004194 discloses that tacrolimus may be used for the treatment of certain disorders. However, WO 2011/004194 did not disclose that a dose different from conventional immunosuppressant doses of tacrolimus should be employed to treat such diseases.

WO 00/15208 discloses that immunosuppressant's may be used to treat matrix metalloproteinase production inhibitors mediated diseases caused by gelatinase and/or collagenase diseases. Many conditions were named from arthritis to tumor growth but fat accumulation was not mentioned. The immunosuppressants included tacrolimus, and the daily dose for chronic administration for all conditions was 0.1 to 0.3 mg/kg/day (thus 7 to 21 mgs for a 70 kg person which would be an immunosuppressant dose).

WO 2000/15208 discloses that tacrolimus may be used for the treatment of certain diseases and notes that the daily dose for chronic use is from 0.1 mg/kg to 30 mg/kg so that for a 70 kg person the daily dose would be 7.5 mg to 210 mg. This range is at least as high as the normal dose range suggested for use of tacrolimus as an immunosuppressant. US 2004/007767 relates to the use of a modified tacrolimus having a methyl group at C₂₁ instead of the propenyl group present in tacrolimus and this also discloses that the daily dose for chronic use is from 0.1 mg/kg to 30 mg/kg.

US 2010/0081681 and US 2013/0102569 disclose that inhibitors of TOR such as rapamycin and analogues may be used to inhibit age related diseases and mentions the immunosuppressive effects of rapamycin, cyclosporine A and tacrolimus. The experimental data was limited to rapamycin and no suggestion was made that doses could be employed in therapy which were less than immunosuppressant doses.

WO 2010/056754 disclosed microcapsulated inhibitors of mTOR, especially rapamycin, which could be used for a range of age related disorders. Individual doses were disclosed which were within the range 0.001 mg to 100 mg or even higher and particularised dose ranges of 5 mg/kg to 100 mg/kg were noted. Only effects of rapamycin were exemplified. It is known that rapamycin use can lead to improvement of glucose control and diabetes.

As a result of studies performed by the Applicant, it has surprisingly been discovered that tacrolimus at a very low dose can be beneficial in treating adverse fat accumulation and associated conditions from that involved in immunosuppression. This is because at this very low dose range tacrolimus accesses a different mechanism in order to lead its newly discovered beneficial effects. It appears that this mechanism is not brought into play when higher doses of tacrolimus are employed.

Studies performed by the Applicant suggest that tacrolimus and its close structural analogues do not produce the beneficial effect described herein through any mechanism related to those which lead to immunosuppressant properties. Studies indicate that tacrolimus and its close structural analogues at certain specific concentrations acts by modulating certain genes. Without wishing to be limited to any mechanism of action, it is postulated that such epigenetic effects play a part in how tacrolimus acts to reduce adverse fat accumulation and/or treat sarcopenia and/or treat metabolic syndrome and its components. This appears to lead to different levels of oxidative enzymes which can lead to a reduction in oxidative stress.

### BRIEF SUMMARY OF THE INVENTION

The present invention provides a method of treatment of one or more conditions in a human in need thereof which comprises administering not more than once a day an effective amount of tacrolimus or a close structural analogue thereof which amount does not cause immunosuppression and which produces a whole blood trough level of tacrolimus or its close structural analogue of at least 0.05 ng/mL, wherein the one or more conditions is one or more of: (i) adverse fat accumulation; (ii) sarcopenia; (iii) hyperglycaemia or type II diabetes; (iv) high cholesterol; (v) high triglycerides; or (vi) metabolic syndrome.

Aptly the invention employs once a day dose of 0.0007 mg/kg to 0.03 mg/kg of such agents.

The invention can lead to a reduction in oxidative stress and/or epigenetic modification which can lead to loss in accumulated fat, a reduction in high cholesterol levels, a reduction in triglyceride levels and/or the other conditions set out above.

### DESCRIPTION OF THE FIGURES

FIG. 1 shows the extension on lifespan profile of S. Cerevisiae resulting from the administration of tacrolimus.

FIG. 2 shows the extension on worm lifespan in Bu8 background caused by tacrolimus.

FIG. 3 shows the delay in the time at which maximal lipid occurs in C. elegans caused by tacrolimus.

FIG. 4 illustrates the model employed which demonstrated the reduction caused by tacrolimus on aging rats.

FIG. 5 shows the reduction in body weights of aging rats caused by the administration of tacrolimus.

FIG. 6 presents data showing the effect of tacrolimus on fat metabolites and various biochemical markers.

FIG. 7 shows the effect on worms lifespan of tacrolimus and its close structural analogue ascomycin.

FIG. 8 shows the effect on worms lifespan of tacrolimus and its close structural analogue dihydrotacrolimus.

FIG. 9 shows the dose response of tacrolimus on Bus8 worm healthspan and median lifespan.

FIG. 10 shows the dose response of tacrolimus on Bus5 worm healthspan and median lifespan.

### DETAILED DESCRIPTION OF THE INVENTION

Accordingly the present invention provides a method of treating adverse fat accumulation in a human in need thereof which comprises administering not more than once a day an effective amount of tacrolimus or a close structural analogue thereof which amount does not cause immunosuppression and which produces a whole blood trough level of tacrolimus or its close structural analogue of at least 0.05 ng/mL.

Similarly, the present invention provides tacrolimus or a close structural analogue thereof for use in treating adverse fat accumulation by administering not more than once a day a dose which does not cause immunosuppression and which produces a whole blood trough level of tacrolimus or close structural analogue thereof of at least 0.05 ng/mL.

Similarly, the present invention provides tacrolimus or a close structural analogue thereof for use in the manufacture of a medicament for treating adverse fat accumulation by administration not more than once a day which medicament does not cause immunosuppression and produces a whole blood trough level of tacrolimus or close structural analogue thereof of at least 0.05 ng/mL.

The trough whole blood level may aptly be at least 0.075 ng/mL, for example at least 0.2 ng/mL such as at least 0.3 ng/mL.

In order to avoid immunosuppression the trough blood level will be less than one third that which is considered immunosuppressant when tacrolimus is used as an immunosuppressant. Generally, this means less than one third of the 4 ng/mL employed in order to prevent transplant rejection i.e. not more than 1.3 ng/mL.

It is believed that to benefit most from the therapeutic window offered by tacrolimus or its close structural analogue the whole blood trough level should be less than 1.2 ng/mL, for example less than 1.1 ng/mL such as 1.0 ng/mL.

The dose of tacrolimus or a close structural analogue thereof employed is therefore advantageously less than that used to produce its clinical immunosuppressant effects when treating organ rejection or diseases such as arthritis or myasthenia gravis.

The mechanism by which tacrolimus or a close structural analogue provides it desirable effect in the present invention is not related to the mechanism by which it achieves immunosuppression. Indeed, use at conventional immunosuppressant doses are believed to be at best poorly effective. Tacrolimus and its close structural analogues have now been found to have a dose response with respect to the conditions disclosed herein curve with a pronounced bell shape with an upper cut off well below conventional immunosuppressant doses.

It is believed that treatment with the disclosed dose of tacrolimus or its close structural analogue in this way induces an epigenetic change which reduces oxidative stress (possibly through effects on oxidative enzymes such as SOD1 and SOD2). This enhances the ability of muscle cell to maintain basal metabolic rate which may be a cause of the reduction in fat accumulation.

It is an advantage of this invention that unlike other methods of fat reduction, for example by calorific limitation, loss of desirable lean muscle mass is not an accompanying significant side effect.

Use of tacrolimus (or close structural analogue) at the doses disclosed herein, can lead to a significant initial reduction in fat accumulation in a patient towards that associated with that patient in younger life. Subsequent administration of tacrolimus (or close structural analogue) can then be employed to maintain or assist in the maintenance of that reduced fat accumulation.

The use therefore can lead to an apparently more youthful silhouette. Sometimes this is accompanied by a minor loss in lean tissue as this may also be associated with the patients more youthful appearing condition (but not muscle wastage, as occurs in sarcopenia).

Hence, this invention is particularly apt for application to older patients where age related fat accumulation has occurred as a result of reduction in metabolic rate. Hence the invention has particular relevance for those over the age of 30, more so for those over the age of 40, yet more so for those over the age of 50.

A further benefit is that the muscle cells enhanced ability to deal with oxidative stress produced by treatment according to this invention may help prevent muscle atrophy and promote regeneration. Not only does this reduce the cells need to store excess calorific intake as fat, it helps counter age related muscle loss.

Hence, the present invention also plays a role in treating sarcopenia.

Accordingly the present invention provides a method of treating sarcopenia which comprises administering to a human in need thereof not more than once a day an effective amount of tacrolimus or a close structural analogue thereof which does not cause immunosuppression and which produces a trough whole blood level of tacrolimus or its close structural analogue of at least 0.05 ng/mL.

Similarly, the present invention provides tacrolimus or a close structural analogue thereof for treating sarcopenia, wherein tacrolimus or its close structural analogue is administered not more than once a day in a dose which does not cause immunosuppression and which produces a trough whole blood level of tacrolimus or its close structural analogue of at least 0.05 ng/mL.

Similarly, the present invention provides the use of tacrolimus or its close structural analogue in the manufacture of a medicament for treating sarcopenia which medicament contains an amount of tacrolimus or its close structural analogue that when administered once per day dose not cause immunosuppression and which has a trough whole blood level of at least 0.05 ng/mL.

If desired the invention permits treatment of adverse fat accumulation and sarcopenia by administration of the same dose of tacrolimus or its close structural analogue.

When tacrolimus is used at doses sufficient to cause immunosuppression it can lead to hyperglycaemia and type II diabetes. It is therefore particularly surprising that when employed in very low doses it can be used to stabilise glucose levels and treat type II diabetes.

Accordingly, the present invention provides a method of treating type II diabetes which comprises administering to a human in need thereof not more than once a day an effective amount of tacrolimus or a close structural analogue thereof which does not cause immunosuppression and which produces a trough whole blood level of tacrolimus or its close structural analogue of at least 0.05 ng/mL.

Similarly, the present invention provides a tacrolimus or a close structural analogue thereof for use in the treatment of type II diabetes in an amount which does not cause immunosuppression and which produces a trough whole blood level of tacrolimus or its close structural analogue of at least 0.5 ng/mL.

Similarly, the present invention provides tacrolimus or a close structural analogue thereof for use in the manufacture of a medicament which contains an amount of tacrolimus or its close structural analogue that when administered once a day does not cause immunosuppression and which has a trough whole blood level of at least 0.05 ng/mL.

The treatment of type II diabetes may be carried out on patients who are being treated for adverse fat accumulation and/or sarcopenia as well as on those who do not. It is however, a particular advantage of the present invention that the patient can be treated with the same very low dose of tacrolimus or its close structural analogue used to treat adverse fat accumulation and/or sarcopenia.

It has also been found that very low dose of tacrolimus or its close structural analogue has a beneficial effect on blood fat levels and particularly on the blood levels of cholesterol and of triglycerides.

Accordingly, the present invention provides a method of treating high blood fat levels which comprises administering to a human in need thereof not more than once a day an effective amount of tacrolimus or a close structural analogue thereof which does not cause immunosuppression and which produces a trough whole blood level of tacrolimus or its close structural analogue of at least 0.05 ng/mL.

Similarly, the present invention provides a tacrolimus or a close structural analogue thereof for use in the treatment of high blood fat levels in an amount which does not cause immunosuppression and which produces a trough whole blood level of tacrolimus or its close structural analogue of at least 0.5 ng/mL.

Similarly, the present invention provides tacrolimus or a close structural analogue thereof for use in the manufacture of a medicament which contains an amount of tacrolimus or its close structural analogue that when administered once a day does not cause immunosuppression and which has a trough whole blood level of at least 0.05 ng/mL.

Aptly a blood fat to be treated is cholesterol. Aptly a blood fat to be treated is triglyceride. Aptly the blood fats to be treated are cholesterol and triglyceride.

It is a further advantage of the invention that patients being treated for high levels of either or both of cholesterol and/or triglyceride may simultaneously be treated for one or more of adverse fat accumulation, sarcopenia and type II diabetes.

Metabolic syndrome is a condition where a patient (who generally is one with adverse fat accumulation and who frequently is obese) presents with type II diabetes (or poor control of glucose levels which predispose to type II diabetes) accompanied by high blood fat levels. This condition can lead to major clinical events such as stroke, myocardial infarction, arteriosclerosis and the like. It is therefore important to treat such patients to alleviate the symptoms of metabolic syndrome. Since treatment with a low dose of tacrolimus as described herein can aid in fat reduction and in the control of type II diabetes and in the control of elevated cholesterol and triglyceride levels, such treatment is suitable for helping patients with metabolic syndrome.

Accordingly, the present invention provides a method of treating metabolic syndrome which comprises administering to a human in need thereof not more than once a day an effective amount of tacrolimus or a close structural analogue thereof which does not cause immunosuppression and which produces a trough whole blood level of tacrolimus or its close structural analogue of at least 0.05 ng/mL.

Similarly, the present invention provides a tacrolimus or a close structural analogue thereof for use in the treatment of metabolic syndrome in an amount which does not cause immunosuppression and which produces a trough whole blood level of tacrolimus or its close structural analogue of at least 0.5 ng/mL.

Similarly, the present invention provides tacrolimus or a close structural analogue thereof for use in the manufacture of a medicament for the treatment of metabolic syndrome which contains an amount of tacrolimus or its close structural analogue that when administered once a day does not cause immunosuppression and which has a trough whole blood level of at least 0.05 ng/mL.

The adverse accumulation of fat and other conditions described herein may be that which occurs as part of aging. Thus, this method of this invention may with advantage be applied to persons over the age of 40, 50, 60 or 70 years (although those of fewer years may also be benefitted).

It is an advantage of this invention in that it may assist in maintaining a healthy weight in older people.

This invention may be of particular usefulness in the treatment of the clinically obese where reduction in body fat is often considered to be of major clinical importance, for example where the body mass index (BMI) is greater than 40.

This invention will similarly be of considerable usefulness where the patient is considered over fat, for example with a BMI of 25 to 40.

It is an advantage of the method and use of this invention that reduction in adverse fat accumulation occurs without muscle wastage. Indeed, lean muscle mass may improve at the same time as fat is lost. To aid in this desirable effect adequate protein should be present in the patient's diet. It is possible that resistance exercise could also be of additional benefit to the patient. Similarly the patient should be encouraged to not eat more calories than needed to maintain healthy body weight although dietary restriction that would normally lead to weight loss is not needed. The increase in lean muscle which may occur has the benefit of increasing basal metabolic rate which in turn aids in reduction of adverse fat levels.

It is therefore apparent that the methods and uses of this invention may with particular benefit be applied to patients who are over 40 years old, aptly over 50 years old, more aptly over 60 years old such as patients who are over 70 years old.

The trough whole blood level may aptly be at least 0.1 ng/mL, for example at least 0.2 ng/mL.

In order to avoid immunosuppression the trough blood level will be less than one quarter that which is considered immunosuppressant when tacrolimus is used as an immunosuppressant. Generally, this means less than one third of the 4 ng/mL employed in order to prevent transplant rejection i.e. not more than 1.3 ng/mL.

It is believed that to benefit most from the therapeutic window offered by tacrolimus or its close structural analogue the trough whole blood trough level should be less than 1.2 ng/mL, for example less than 1.1 ng/mL.

The treatment of this invention may be used prophylactically or therapeutically.

It is presently preferred to employ tacrolimus in this invention. However, it is also apt to employ a close structural analogue of tacrolimus. Such analogues retain the structure of tacrolimus from C₁ to C₁₇, C₁₉, C₂₀ and C₂₂ to C₃₄ but permit modifications at C₁₈ and/or C₂₁. Such modifications include permitting one hydrogen atom on either of C₁₈ or C₂₁, to be substituted by a C₁₋₄ alkyl, C₂₋₄ alkenyl, hydroxyl or methyoxyl group and also include replacing the C₂₁ propenyl group by a hydrogen atom, C₁₋₆ alkyl group, other C₂₋₆ alkenyl group, a hydroxyl group or a methoxyl group. Certain apt compounds include analogues of tacrolimus where the C₂₁ position is modified, for example C₂₁ propenyl group is replaced by a methyl group, an ethyl group or a propyl group. Other apt compounds include analogues where a C₁₈ hydrogen atom is replaced by a hydroxyl group. Certain of these compounds are less immunosuppressant than tacrolimus, for example the analogue wherein the C₂₁ propenyl group is replaced by a methyl group or the analogue wherein a C₁₈ hydrogen is replaced by a hydroxyl group (for example wherein the C₂₁ propenyl group is unchanged or replaced by a methyl, ethyl or propyl group). U.S. Pat. No. 5,376,663 discloses process for the preparation of analogues of tacrolimus. The C₂₁ propenyl group of tacrolimus may also be replaced as a fenor C₁₋₆ alkyl group such as a 2-fluoroethyl group or by a different propenyl group such as a 1-methyl ethenyl group.

The term “does not cause immunosuppression” indicates that major side effects of immunosuppression do not normally occur. This results from a dose of tacrolimus or a close structural analogue being employed that does not substantially depress TNFα levels in the patient. It is believed that a dose of less than 1.2 mg per day of such compounds in a 70 kg adult (pro-rata for other body weights) may be considered not to lead to immunosuppression.

However, the maximum daily dose of tacrolimus or a close structural analogue thereof that will be employed for a 70 kg patient will be to maximise benefit of the treatments disclosed herein will be a dose of not more than 0.9 mg and favourably of not more than 0.75 mg will be employed to a 70 kg patient (pro-rata for other body weights) will be employed on any day give a wider separation between the desired effects and side effects.

Studies suggest that in some cases (that because of the bell shaped response curve that results from the epigenetic mechanism) it may be advantageous to employ a dose of not more than 0.6 mg per day for a 70 kg patient (and pro-rata for other weights), for example less than 0.5 mg of tacrolimus or a close structural analogue thereof per day for a 70 kg patient (and pro-rata for other weights).

The Applicant's studies suggest that a dose of not less than 0.05 mg per day of tacrolimus or a close structural analogue will be apt and a dose of not less than 0.1 mg per day may be favoured in some cases, for example a dose of not less than 0.15 mg per day may be employed.

Hence, apt daily doses for a 70 kg patient (with doses pro-rata for other weights) include 0.05 mg, 0.075 mg, 0.1 mg, 0.125 mg, 0.15 mg, 0.175 mg, 0.2 mg, 0.225 mg, 0.25 mg, 0.275 mg, 0.3 mg, 0.325 mg, 0.35 mg, 0.375 mg, 0.4 mg, 0.425 mg, 0.45 mg, 0.475 mg, 0.5 mg, 0.525 mg, 0.55 mg, 0.575 mg and 0.6 mg of tacrolimus or a close structural analogue thereof (for example of tacrolimus).

Because of the epigenetic mechanism of action of tacrolimus and its close structural analogues by which it provides its beneficial effects in treating disease characterised by protein aggregate deposition in neuronal cells, beneficial results may be obtained by dosing frequency of less than once per day, for example once every 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days or more. Particularly suitable intervals for ease of patient use apart from daily may include on alternative days, once a week, once every 10 days, three times a month, once a fortnight or once a month.

From the preceding commentary on doses, the skilled person will understand that the present invention provides a method of treating any one or more of the conditions referred to hereinbefore which comprises administering to a human in need thereof not more than once a day an effective amount of tacrolimus or a close structural analogue thereof wherein the dose is from 0.007 mg/kg to 0.03 mg/kg.

Similarly, the present invention provides tacrolimus or a close structural analogue thereof for treating any one or more of the conditions referred to hereinbefore wherein tacrolimus or its close structural analogue is administered not more than once a day at a dose of 0.007 mg/kg to 0.03 mg/kg.

Similarly the present invention provides the use of tacrolimus or a close structural analogue in the manufacture of a medicament for treating any one or more of the conditions referred to hereinbefore by administration not more than once a day wherein the medicament contains 0.007 mg/kg to 0.03 mg/kg of tacrolimus or its close structural analogue.

Generally not more than 0.1 mg/kg of tacrolimus or its close structural analogue will be employed and aptly not more than 0.085 mg/kg such as 0.007 mg/kg will be employed.

Generally more than 0.0014 mg/kg of tacrolimus or its close structural analogue, for example more than 0.002 mg/kg will be employed.

It is presently considered particularly suitable to employ from 0.0014 mg/kg to 0.0085 mg/kg, for example from 0.002 mg/kg to 0.007 mg/kg of tacrolimus or its close structural analogue.

In such uses tacrolimus may be aptly employed.

It will be appreciated that such doses are very different from doses employed in patients to provide immunosuppression.

The present invention provides a method of treating any one or more of the conditions referred to hereinbefore which comprises administering to a human in need thereof by administration not more than once a day of an effective amount of tacrolimus or a close structural analogue to effect epigenetic modification which leads to a reduction in oxidative stress, for example by modification of SOD1 and/or SOD2 expression.

Similarly, the present invention provides tacrolimus or a close structural analogue thereof for use in treating any one or more of the conditions referred to hereinbefore by administration not more than once a day of an amount which effects epigenetic modification which leads to a reduction in oxidative stress, for example by modification of SOD1 and/or SOD2 expression.

Similarly, the present invention provides the use of tacrolimus or a close structural analogue thereof in the manufacture of a medicament for the treatment of any one or more of the conditions referred to hereinbefore by administration not more than once per day which medicament contains an amount of tacrolimus or close structural analogue thereof which effects epigenetic modification which leads to a reduction in oxidative stress, for example by modification of SOD1 and/or SOD2 expression.

The doses employed and the dose schedules may be as hereinbefore indicated. The presently preferred compound for such use is tacrolimus.

In a further aspect the present invention provides a unit dose pharmaceutical composition containing 0.05 mg to 0.8 mg of tacrolimus or a close structural analogue thereof and a pharmaceutically acceptable carrier therefor for use in the treatment of any one or more of the conditions referred to hereinbefore (that is treating adverse fat accumulation, and/or sarcopenia, and/or type II diabetes, and/or high cholesterol and/or triglyceride levels, and/or metabolic syndrome).

The unit dose for use may contain not more than 0.75 mg, for example a dose of not more than 0.6 mg such as not more than 0.5 mg of tacrolimus or a close structural analogue thereof, preferably tacrolimus.

The unit dose may contain not less than 0.05 mg, favourably not less than 0.1 mg, for example not less than 0.15 mg of tacrolimus or a close structural analogue thereof, preferably tacrolimus.

Such unit doses aptly employ tacrolimus.

Hence favoured unit doses for such use may contain 0.1 mg to 0.6 mg of tacrolimus, for example 0.1 mg to 0.5 mg tacrolimus.

The tacrolimus or its close structural analogue may be as solvates such as hydrates or alcoholates. Aptly tacrolimus is employed as a hydrate such as the monohydrate (when weights are referred to herein they do not include the weight of the solvating molecule).

Unit doses may contain any of the specific amounts set forth hereinbefore.

At present it is preferred that the unit dose will contain tacrolimus, for example as tacrolimus monohydrate.

If desired an existing commercial product such as Prograf® may be purchased and its contents divided to produce the desired dose which may then be placed into a hard gelatin capsule for oral administration.

The unit dose may be suitable for administration by injection but it is preferred that the unit dose is suitable for administration via the mouth. The unit dose may be liquid, for example a solution or suspension in a container, but it is considered preferable that the unit dose in non-liquid. Suitable solid unit dosage forms include tablets and capsules of which capsules are more apt. The skilled pharmaceutical chemist has decades of research into pharmaceutical compositions containing tacrolimus upon which to base preparation of unit doses.

In some cases, a pharmaceutical composition is formulated for oral administration. In some cases, the composition comprises a suspension of a compound in a suitable vehicle. Non-limiting examples of vehicles for oral administration include phosphate-buffered saline (PBS), 5% dextrose in water (D5W) and a syrup. The composition may be formulated to stabilize the consistency of a dose over a period of storage and administration. In some cases the composition may be a solution of the active compound dissolved in a diluent such as water, saline, and buffers optionally containing an acceptable solubilizing agent. In favoured form, the composition comprises a solid dosage form. In some cases, the solid dosage form comprises a capsule, a caplet, a lozenge, a sachet, or a tablet. In some cases, the solid dosage form is a liquid-filled dosage form. In some cases, the solid dosage form is a solid-filled dosage form. In some cases, the solid dosage form is a solid-filled tablet, capsule, or caplet. In some cases, the solid-filled dosage form is a powder-filled dosage form. In some cases, the solid dosage form comprises a compound in the form of micronized particles, granules or microcapsulated agent. In some cases, the composition comprises an emulsion which may contain a surfactant. In some cases, the solid dosage form comprises one or more of lactose, sorbitol, maltitol, mannitol, cornstarch, potato starch, microcrystalline cellulose, hydroxypropyl cellulose, acacia, gelatin, colloidal silicon dioxide, croscarmellose sodium, talc, magnesium stearate, stearic acid, pharmaceutically-acceptable excipients, colorants, diluents, buffering agents, moistening agents, preservatives, flavoring agents, carriers, and binders. In some cases, the solid dosage form comprises one or more materials that facilitate manufacturing, processing or stability of the solid dosage form or a flavoring agent.

When considering such formulation the skilled worker may call upon the many years experience in the art of formulating tacrolimus and of formulating low dose pharmaceuticals in general.

The present invention also provides a method of cosmetic fat reduction which comprises employing a very low dose of tacrolimus or close structural analogue as hereinbefore defined. The present invention also provides a method of enhanced muscle appearance which comprises employing a very low dose of tacrolimus or close structural as hereinbefore defined. Generally such methods may be applied when not clinically necessary but when a subjects sense of wellbeing or self-worth would benefit from such treatment.

As indicated hereinbefore it is normal and greatly preferred not to administer the tacrolimus or close structural analogue more than once a day. However, in a much less preferred aspect, the present invention also provides methods and uses as hereinbefore defined modified so that tacrolimus or a close structural analogue is administered twice a day (as two divided doses).

In a further aspect the present invention provides a unit dose pharmaceutical composition containing 0.05 mg to 1.2 mg of tacrolimus or a close structural analogue thereof and a pharmaceutically acceptable carrier therefore for use in the treatment of a disease characterized by deposition of adverse fat accumulation or adverse cholesterol levels.

Suitably the oral pharmaceutical composition is for the treatment of adverse accumulation of fat. Suitably the oral pharmaceutical composition is for the treatment of adverse cholesterol levels.

The unit dose may contain not more than 0.9 mg or 0.75 mg, for example a dose of not more than 0.65 mg such as not more than 0.5 mg or not more than 0.45 mg of tacrolimus or a close structural analogue thereof, preferably tacrolimus.

Such unit dose may be for administration as described herein, particularly for oral administration.

The unit dose may contain not less than 0.05 mg, favourably not less than 0.1 mg, for example not less than 0.15 mg of tacrolimus or a close structural analogue thereof, preferably tacrolimus.

Hence, aptly the unit dose may contain from 0.05 mg to 0.9 mg, for example from 0.1 mg to 0.75 mg, for example from 0.15 mg to 0.6 mg or from 0.15 mg to 0.5 or 0.45 mg of tacrolimus. Such unit doses may be adapted for oral administration, for example as a tablet or preferably a capsule.

It is presently envisaged that the favoured daily doses of tacrolimus for the treatment of adverse fat accumulation is from 0.05 to 0.65 mg, for example, 0.1 mg to 0.5 mg, such as 0.15 mg to 0.45 mg, for example, 0.3 mg. Such doses are aptly orally administered, and desirably not more than once per day for example, once a day using the unit doses described herein.

Aptly the invention is employed to reduce sub-dermal fat accumulation. This may be of particular benefit for those seeking cosmetic enhancements, for example for reduction in fat around the trunk, for example over the abdomen. Aptly this invention is employed to reduce fat within the body cavity, for example, that associated with internal organs such as the heart, liver and intestines which is known to cause adverse health effects.

Pharmacokinetic analysis has been carried out by the Applicant and the human doses herein based thereon, (in light of the data provided in the Examples).

The Examples herein show that the effects of tacrolimus occurs in yeasts, worms and mammals indicating its ability to effect oxidative stress is evolutionarily conserved. Hence, human disease will be treatable with tacrolimus (and close structural analogues) in the same way as with the other species including the mouse.

The following Examples illustrate the invention. Reference herein to RDC5 is to tacrolimus.

### REFERENCES

Ahima, R. S. (2009). Connecting obesity, aging and diabetes. Nature medicine, 15(9), 996-7. doi: 10.1038/nm 0909-996
Ferrucci, L., & Studenski, S. (2009). Diabetes, muscles, and the myth of Ulysses' bow. Diabetes care, 32(11), 2136-7. doi:10.2337/dc09-1592
Goodpaster, B. H., Leland Thaete, F., Simoneau, J.-A., & Kelley, D. E. (1997). Subcutaneous Abdominal Fat and Thigh Muscle Composition Predict Insulin Sensitivity Independently of Visceral Fat. Diabetes, 46(10), 1579-1585. doi:10.2337/diacare.46.10.1579
Leong, K. S., & Wilding, J. P. (1999). Obesity and diabetes. Best Practice & Research Clinical Endocrinology & Metabolism, 13(2), 221-237. doi:10.1053/beem.1999.0017
Malandrucco, I., Pasqualetti, P., Giordani, I., Manfellotto, D., De Marco, F., Alegiani, F., Sidoti, A. M., et al. (2012). Very-low-calorie diet: a quick therapeutic tool to improve β cell function in morbidly obese patients with type 2 diabetes. The American journal of clinical nutrition, 95(3), 609-13. Retrieved from http://www.ncbi.nlm.nih.gov/pubmed/22318758
Seki, Y., Williams, L., Vuguin, P. M., & Charron, M. J. (2012). Minireview: Epigenetic programming of diabetes and obesity: animal models. Endocrinology, 153(3), 1031-8. doi:10.1210/en.2011-1805
Zheng, J., & Greenway, F. L. (2012). Caenorhabditis elegans as a model for obesity research. International journal of obesity (2005), 36(2), 186-94. doi:10.1038/ijo.2011.93

### Example 1

### Tacrolimus Delays Ageing in Yeast and Worms

The yeast bioscreen measures the Chronological Lifespan Profile of an ageing yeast cultures. The median lifespan is defined as the time at which 50% of a given population has died. Relative viability (expressed in the below chart) compares the effect of treatment on a cultures viability as a fraction of control vehicle treated cultures. RDC5 was identified as being able to extend the chronological lifespan profile of S. Cerevisiae without effecting growth rate

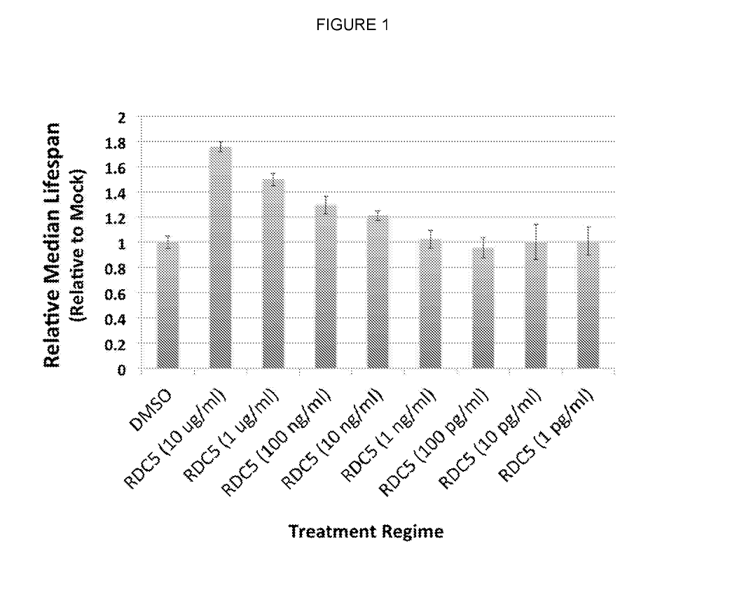

RDC5 treated yeast cultures have a significantly longer median lifespan than control treated cultures. RDC5 is thus believed to be able to delay ageing and the onset of age-related diseases such as unwanted fat accumulation. The effect on life span is shown in FIG. 1.

Worm cultures of at least 80 worms were manually assessed daily for the duration of the lifespan. RDC5 is also able to extend the Healthspan and Median lifespan of populations of worms as well as yeast.

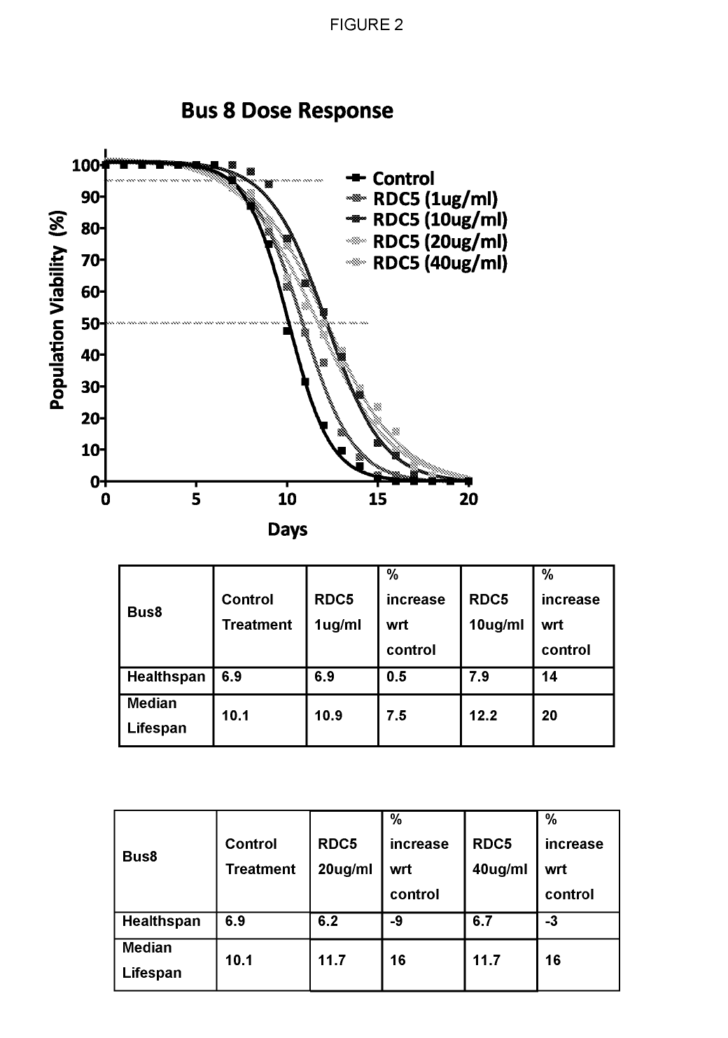

Effect of Tacrolimus on Worm Lifespan in Bus8 Background is shown in FIG. 2.

The low dose of 10 ug/ml is the most efficacious at extending Healthspan in Bus8 worms.

### Example 2

### Effect on Lipid Accumulation Worms

Tacrolimus is able to delay the age dependent increase in fat accumulation in C. elegans. Worms are a versatile model of obesity ref: (Zheng & Greenway, 2012)

Oil-Red-O is a fat-soluble dye used for staining lipids and is quantified by colour light microscopy.

The DMSO (control) treated C. elegans showed a rise in lipid deposits from day 1 to day 4, followed by a plateau period until day 9. In contrast, RDC5 treated worms showed a shallower rise in lipid accumulation, peaking at day 7.

RDC5 delays the time at which maximal lipid accumulation happens from 4-7 days post adulthood. This demonstrates RDC5 is acting to slow fat accumulation and would be of use in treating age-related obesity.

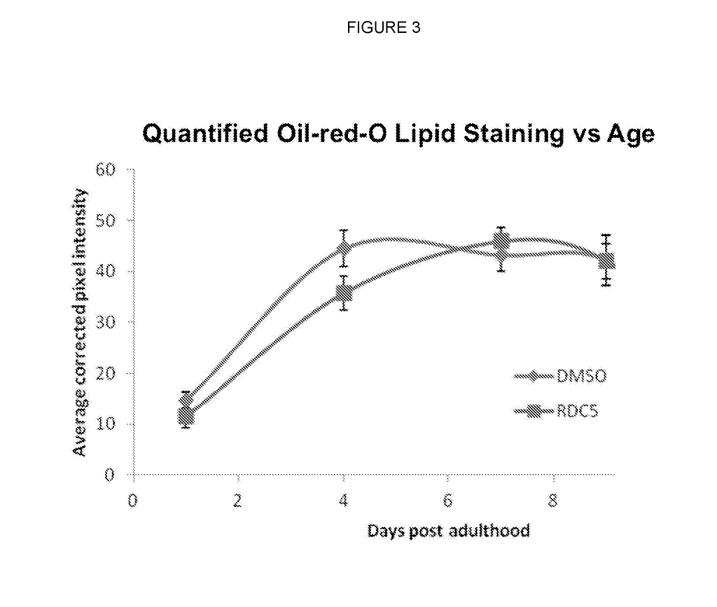

The results are shown in FIG. 3.

### Example 3

### Effect on Obesity in Rat

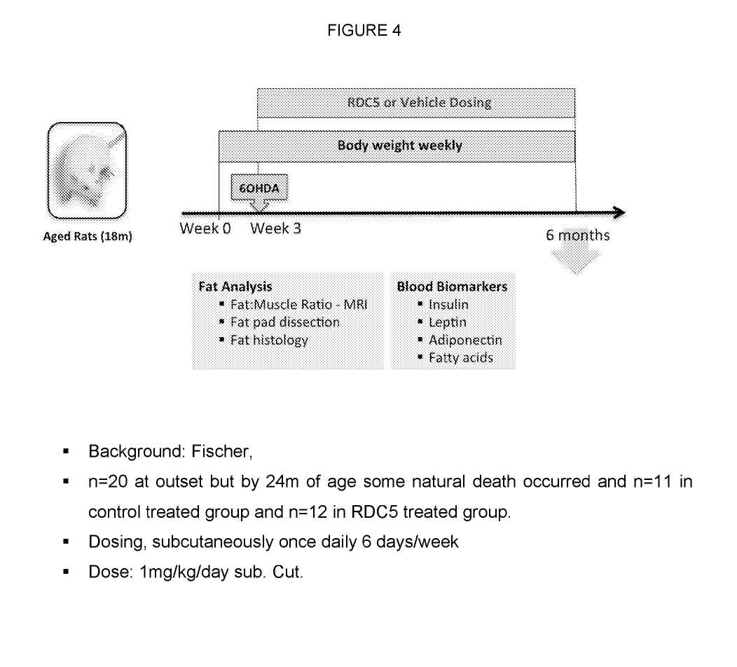

This data describes the effect of RDC5 on age dependent fat increase in rats. Aged rats are known to increase in body weight dramatically in late adulthood. Aged rats were being used as a model in RAT_ND_01 and body weights and tissue samples were taken from the rats in this trial. Rats were 18 m of age at the start of the 6 m trial. The dosage is seen in FIG. 4.

RAT_{—} FAT_01 Body Weight Data

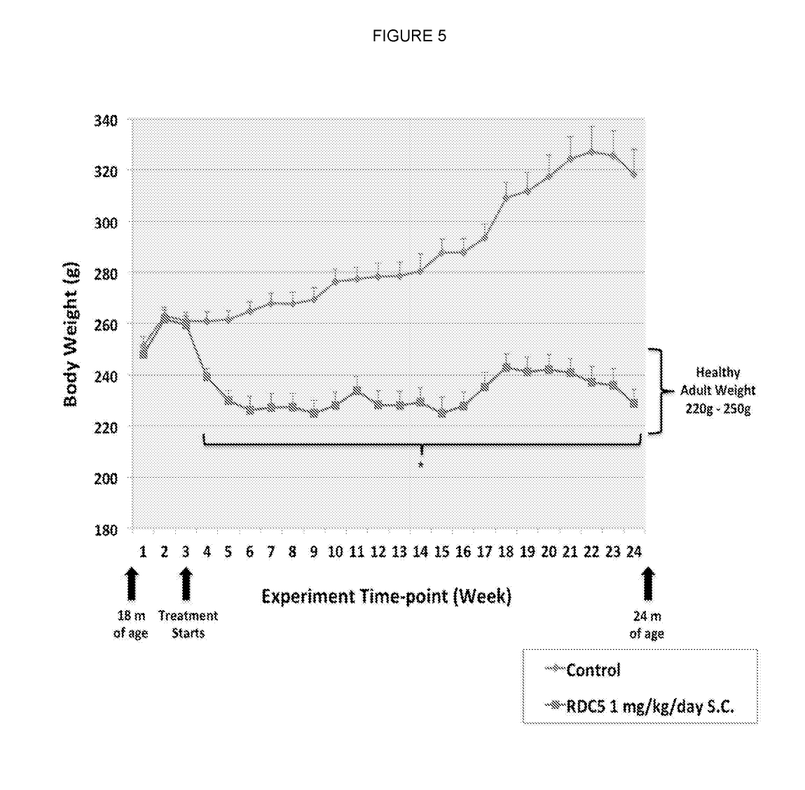

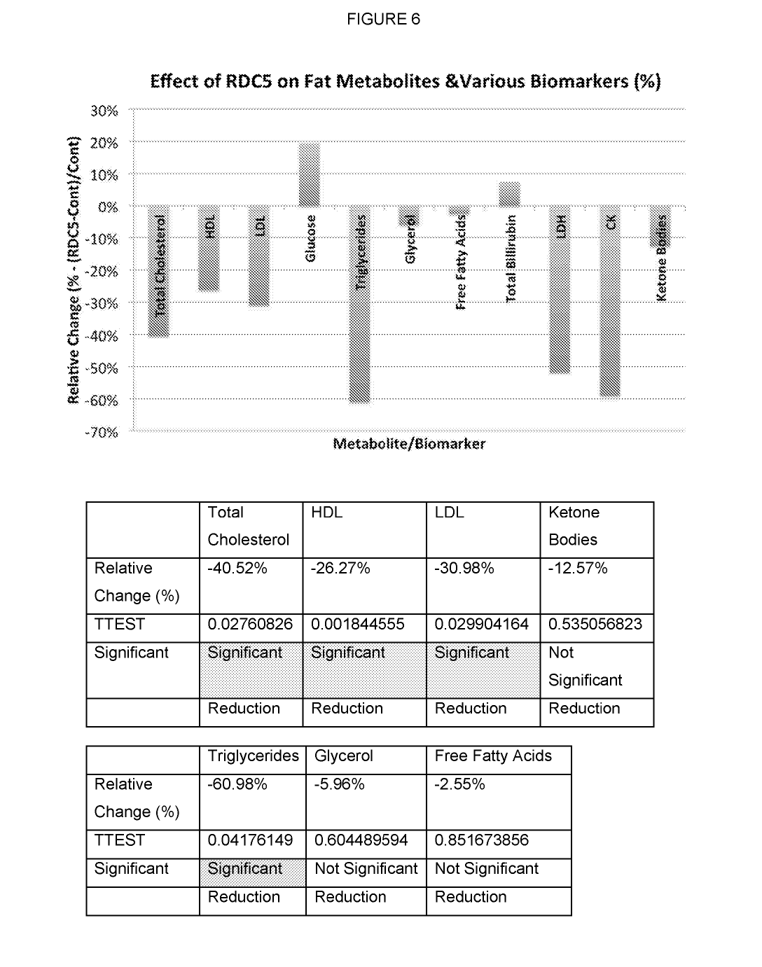

Throughout this study (i.e. from 18 months of age onwards), individual weekly body weight assessments were made for both the control and RDC5 treated group. (Rat body weights are statistically significantly different from week 4 until end of trial. Error bars are Standard Error of the Mean). At the 1 and 2 week time-points, before the onset of treatment, no significant difference between RDC5 and control treatment could be observed. Control treated rat body weight continued to increase over time from approximately 250 g to over 320 g at the 6 month time-point (at 24 months of age). RDC5 treated rats however did not suffer the same levels of age related weight gain. Weight levels dropped initially however not below a healthy age (approx. 230 g) and remained constant (+/−20 g) for the remainder of the study. The result of this was the RDC5 rats weighing significantly less than their control treated age-matched controls from 4 weeks of age onwards and being 28% lighter by the end of the study. The results are shown in FIG. 5.

### Biomarker Data

### Metabolic Biomarkers

A range of techniques has been used to determine the effect RDC5 has on levels of blood biomarkers of obesity and metabolic disorders.

Metabolic biomarkers tested included:
Ketone Bodies—kinetic enzymatic
Free Fatty acids—ACS-ACOD Enzymatic
Glycerol—Calometric
Triglycerides—GPO-POD
HDL Cholesterol—CHO-PAP
LDL Cholesterol—Enzymatic
Total Cholesterol—CHO-PAP

All markers of obesity are reduced (however, glycerol, free fatty acids and ketone bodies reduction is not statistically significant). 6 m RDC5 treatment in rats results in significant reductions in total cholesterol, HDL, LDL, and triglycerides show that RDC5 has a beneficial effect in controlling these components of metabolic syndrome.

### Example 4

Fifty female Fischer rats initially aged 18 months were used in this study. Animals were housed at standard temperature (22±1° C.) in light controlled environment (lights on from 7 am to 8 pm) with ad libitum access to food and water with softer food introduced at 17 weeks to encourage food intake. Half the animals were treated with vehicle (5 ml/kg, s.c.) 6 days a week and half were treated with RDC5 (1 mg/kg, s.c.) 6 days a week. Individual body weights were recorded at baseline and then once a week until the end of the study.

No significant differences in survival of animals at the end of the study was noted (slightly more rats on RDC5 survived).

At 1 and 2 week time points, before onset of treatment, no significant differences between RDC5 treated and control treated groups could be observed. Control treated rat body weight continued to increase over time from approximately 250 g to approximately 320 g at the 6 month time point. Age related weight gain generally indicates an increase in body fat both in absolute terms and % body weight terms.

RDC5 treated rats did not show age related weight gain. Weight levels dropped initially but not to below a healthy weight (approximately 230 g) and remained approximately constant (±20 g) for the remainder of the study. The RDC5 treated rats weighed significantly less than their control treated age-matched controls from three weeks after commencement of the study to its end at which point they weighed 28% lighter (approximately 230 g as opposed to approximately 320 g).

Post mortem MR1 scanning demonstrated fat:lean ratio. FIG. 11 shows exemplar cross sectional images from young, 3 month rats, control treated rats, 24 months old and RDC5 treated rats at 24 months old.

Aged rats display an increase in both sub-cutaneous fat and visceral rat compared to young rats. However, the RDC5 treated rats are seen to possess less sub-cutaneous fat at less visceral fat than control treated rats at 24 months.

The 3 month rats had an average of roughly 20% body fat, control rats at 24 months had an average of roughly 40% body fat and RDC5 treated rats at 24 months had roughly 30% body fat.

FIG. 11 presents MIR data comparing 3 month old untreated animal, 24 month old control treated animal and 24 month old tacrolimus treated animal. The 24 month old tacrolimus treated animal is seen to have sub-cutaneous fat akin to the 3 month old untreated animal and much less fat within the body cavity than the 24 month old control treated animal. This is consistent with a cosmetic improvement (with respect to reduction in sub-cutaneous fat) and a health improvement (with respect to reduction of fat near internal organs) produced by tacrolimus treatment.

The MIR images are marked (areas 1) to show internal fat areas. The sub-cutaneous fat layer is the light grey outer ring only easily seen in the 24 month old untreated control.

### Example 5

The RDC5 treated and control treated animals had their blood plasma profiled at the end of the investigations. The following data is a summary of that observed:

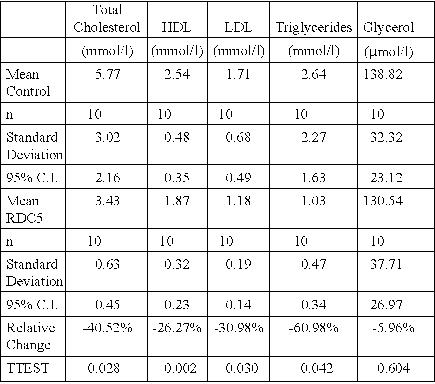

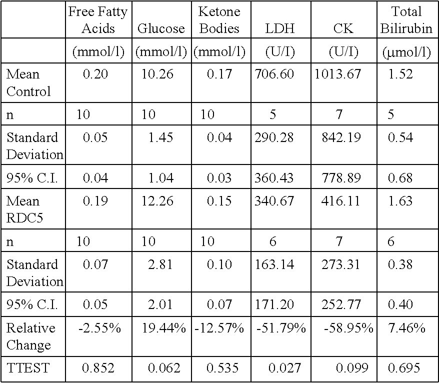

This shows that treatment with the low dose of RDC5 resulted in a beneficial 40% reduction in total cholesterol and a beneficial reduction in triglycerides. This is consistent with the use of the very low dose of RDC5 to be employed in humans providing desirable effects on cholesterol and triglycerides consistent with a reduced likelihood of stroke and adverse effects on the heart.

The 50% reduction in lactate dehydrogenase levels in consistent not only a lack of toxicity at the very dose to be employed but is consistent with improved resistance to muscle loss that can occur in disease states (such as cancer or HIV) in the very aged.

### Example 6

### Effects of Close Structural Analogues of Tacrolimus

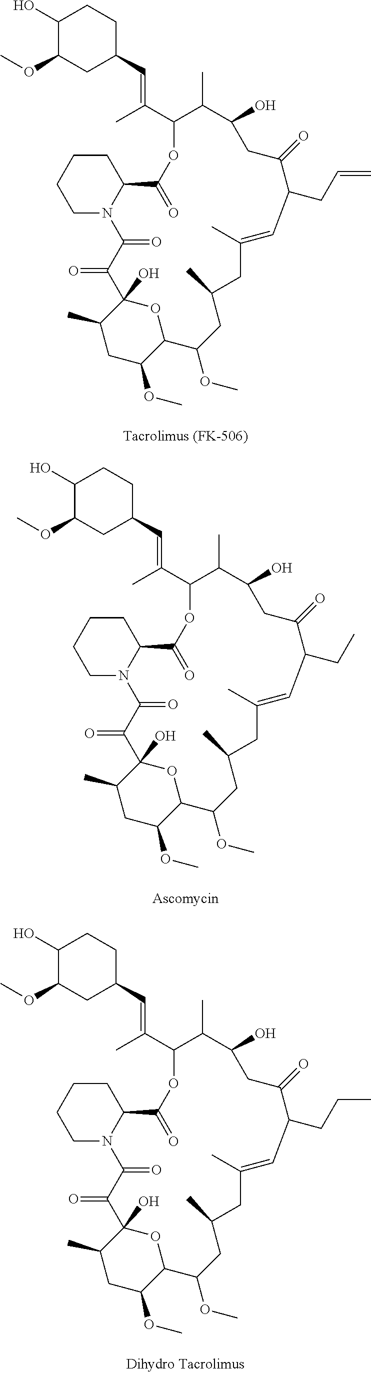

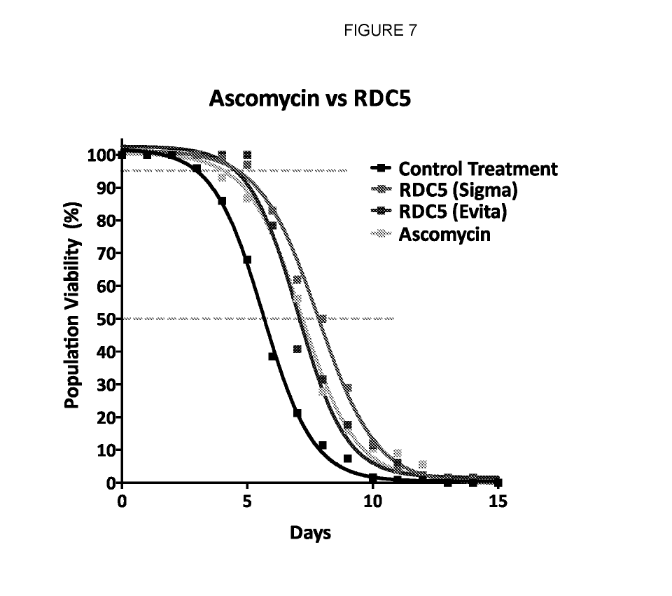

The lifespan plot shown in FIG. 7 shows that Ascomycin (21-ethyl analogue) is able to extend the lifespan of worms as effectively as tacrolimus. This plot in FIG. O also shows that different sources of tacrolimus have varied efficacy in extending lifespan in worms (Sigma vs Evita) possibly due to quality of source.

| | | RDC5 | RDC5 | |
|---|---|---|---|---|
| | Control | (Sigma) | (Evita) | Ascomycin |
| Healthspan | 3.0 | 4.6 | 4.5 | 4.1 |
| Median Lifespan | 5.7 | 7.8 | 7.1 | 7.2 |
| Max Lifespan | 8.7 | 11.2 | 10.3 | 10.6 |

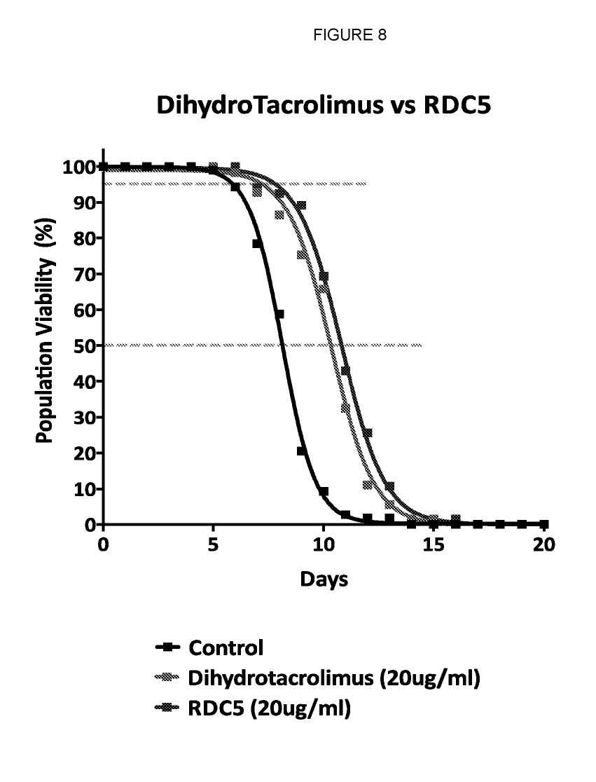

The lifespan plot in FIG. 8 shows that Dihydrotacrolimus (21-propyl analogue) is able to extend the lifespan of worms almost as effectively as tacrolimus. This plot also shows that different sources of RDC5 have varied efficacy in extending lifespan in worms (Sigma vs Evita).

| | Control | Dihydrotacrolimus | RDC5 |
|---|---|---|---|
| Healthspan | 5.9 | 7.3 | 7.8 |
| Median Lifespan | 8.1 | 10.3 | 10.8 |
| Max lifespan | 10.4 | 13.2 | 13.8 |

This data presented in FIGS. 7 and 8 demonstrate that the mechanisms by which very low dose tacrolimus exhibits its effects are also present in close structural analogues such as dihydrotacrolimus and the 21-ethyl analogue of tacrolimus.

### Example 7

### Unit Dose

A hard gelatine capsule was filled with the following composition:

| | Formulation | | Reference |
|---|---|---|---|
| Component | (mg) | Function | Standard |
| RDC5 | 0.30 | Active ingredient | USP |
| Lactose monohydrate | 89.45 | Diluent | Ph Eur |
| Hydroxypropylmethyl | 6.00 | Binder | Ph Eur |
| cellulose | | | |
| Croscarmellose sodium | 4.00 | Super- | Ph Eur |
| | | disintegrant | |
| Magnesium stearate | 0.25 | Lubricant | Ph Eur |
| Ethanol | qs | Binder fluid | |

One oral capsule as above containing 0.3 mg tacrolimus was administered to groups of healthy human volunteers. No significant changes in blood TNF-α levels occurred as a result of the administration. The average trough level of tacrolimus (level after 24 hours of administration of each dose) observed was approximately 220 pg/mL. The average peak level of tacrolimus observed was approximately 3700 pg/mL and the average area under the curve was approximately AUC 0-t=23500 (h*pg/mL).

The above capsules may be used to provide the treatments described hereinbefore.

Use of two such capsules to provide a single dose of 0.6 mg would be expected to result in a trough level of about 440 pg/mL.

### Example 8

### Dose Optimization for Tacrolimus

The facts outlined below shows that RDC5 produces as bell shaped dose response in: worm lifespan extension, response to oxidative stress of Osteoblast cells.

These dose define the efficacious dose range for RDC5.

| | | | % | | % | | % |
|---|---|---|---|---|---|---|---|
| | | | increase | | increase | | increase |
| | Control | RDC5 | wrt | RDC5 | wrt | RDC5 | wrt |
| Bus5 | Treatment | 1 ug/ml | control | 10 ug/ml | control | 40 ug/ml | control |
| Healthspan | 10.93 | 14.55 | 33 | 13.43 | 23 | 10.28 | −6 |
| Median | 15.08 | 18.54 | 23 | 19.03 | 26 | 15.17 | 0.5 |
| Lifespan | | | | | | | |

Lower doses of 1 ug/ml and 10 ug/ml are most efficacious at extending CLS in Bus5 worms. (20 ug/ml contaminated)

| | | | % | | % | | % | | % |
|---|---|---|---|---|---|---|---|---|---|
| | | | increase | | increase | | increase | | increase |
| | Control | RDC5 | wrt | RDC5 | wrt | RDC5 | wrt | RDC5 | wrt |
| Bus8 | Treatment | 1 ug/ml | control | 10 ug/ml | control | 20 ug/ml | control | 40 ug/ml | control |
| Healthspan | 6.87 | 6.90 | 0.5 | 7.85 | 14 | 6.23 | −9 | 6.67 | −3 |
| Median | 10.10 | 10.86 | 7.5 | 12.18 | 20 | 11.70 | 16 | 11.70 | 16 |
| Lifespan | | | | | | | | | |

Low dose of 10 ug/ml is the most efficacious at extending Healthspan in Bus8 worms. Higher doses of 20 ug/ml and 40 ug/ml match the efficacy of 10 ug/ml at Median and Maximal Lifespan extension but not Healthspan.

(Coli food source and FUDR have been ruled out as possibly affecting lifespan.)

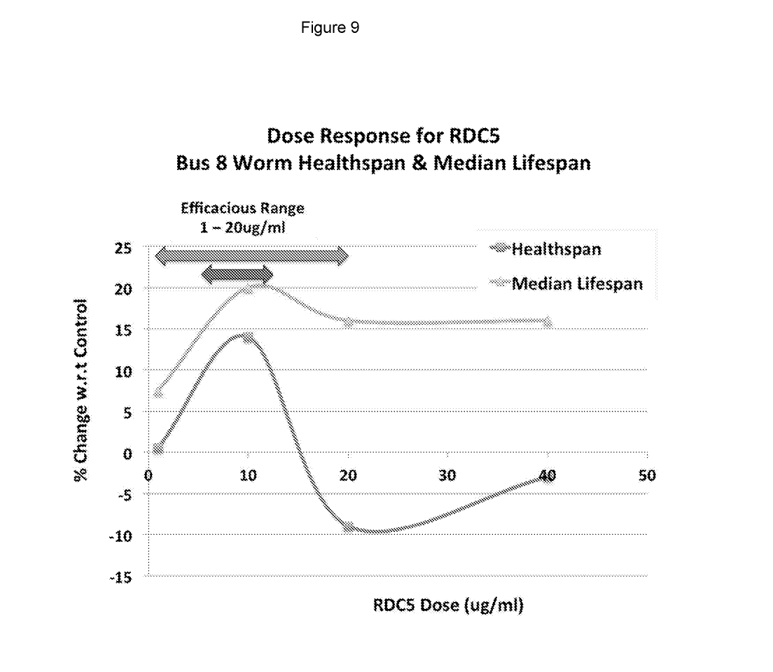

As shown in FIG. 9 the dose response in worms→narrow window of efficacy in CLS at lower end of doses tested, 10 ug/ml is consistently the most efficacious dose for improving healthspan, even lower doses are sometime also effective and high dose of 40 ug/ml is always the least effective.

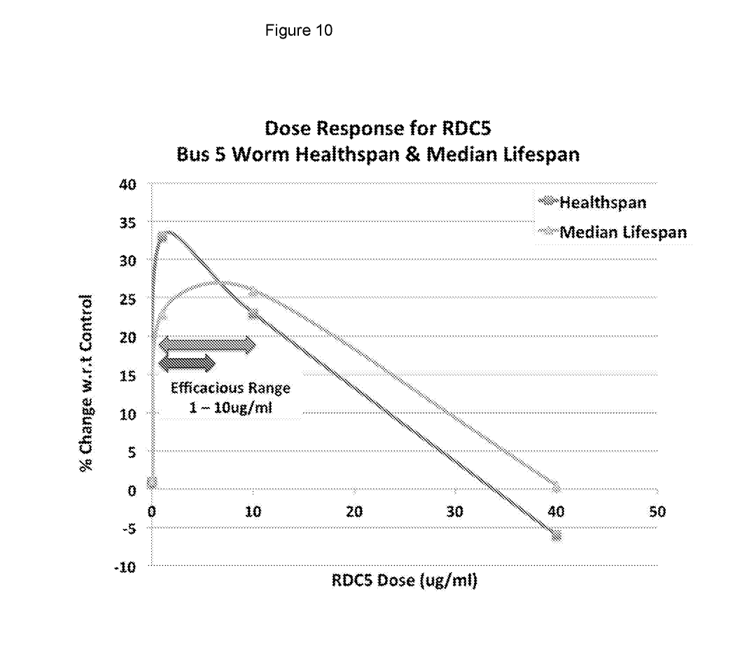

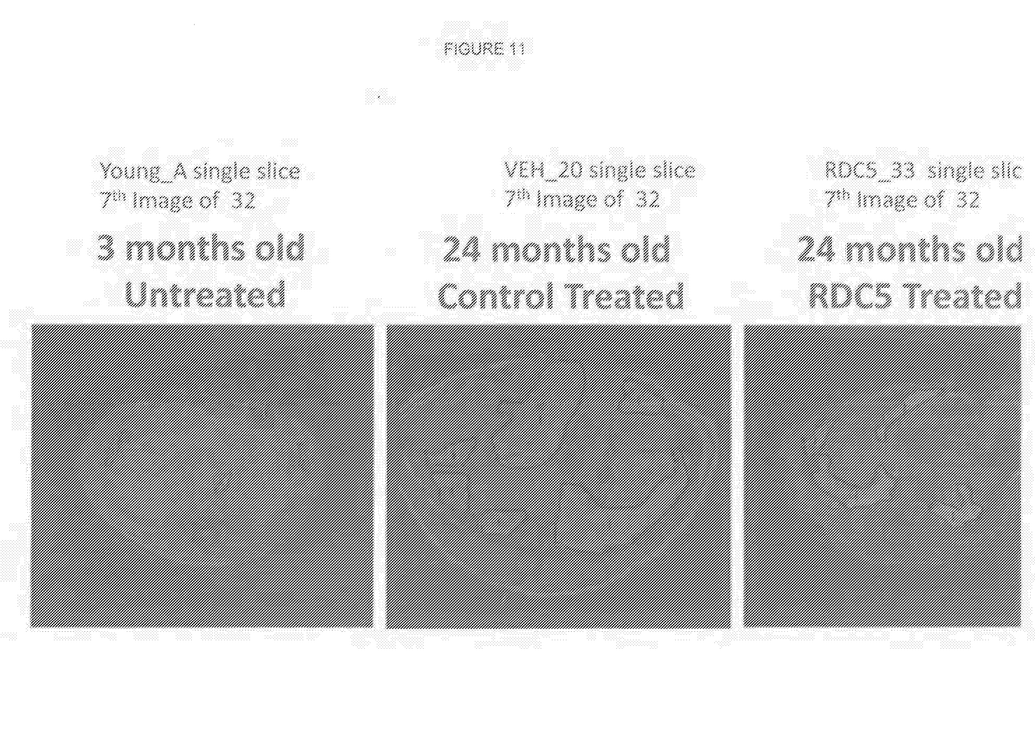

In Bus8 worms a small efficacious dose range for Healthspan, larger range for dose response when considering Median Lifespan, but a efficacy peaking at 10 ug/ml for both Healthspan and Median Lifespan extension. This is shown in FIG. 10.

In Bus8 worms again there is a small efficacious dose range for Healthspan and a larger range for dose response for Median Lifespan. Efficacy peaking at 1 ug/ml for Healthspan extension and 10 ug/ml for Median Lifespan extension.

In further experiments it was determined that dosing on two thirds of days also produced significant increases in lifespan compared to control and that dosing on one third of days produced significant increases in lifespan (except for when dosing was not commenced early in lifespan). This is consistent with the desired therapeutic effects described herein being achieved with less than every day administration.

1-51. (canceled)

52. A pharmaceutical composition comprising Tacrolimus or a close structural analogue thereof, which produces a whole blood trough level of tacrolimus or its close structural analogue of at least 0.05 ng/mL, wherein the Tacrolimus or a close structural analogue is in a therapeutically effective amount for treating one or more conditions selected from the group consisting of:
(i) adverse fat accumulation;
(ii) sarcopenia;
(iii) hyperglycaemia or type II diabetes;
(iv) high cholesterol;
(v) high triglycerides;
(vi) metabolic syndrome; and
(vii) combinations thereof.

53. The pharmaceutical composition of claim 52 where the trough whole blood level produced is at least 0.1 ng/mL.

54. The pharmaceutical composition of claim 52 wherein the trough whole blood level produced is at least 0.2 ng/mL.

55. The pharmaceutical composition of claim 52 wherein the trough whole blood level produced is less than 1.2 ng/mL.

56. The pharmaceutical composition of claim 52 wherein the trough whole blood level produced is less than 1.1 ng/mL.

57. The pharmaceutical composition of claim 52 which comprises tacrolimus.

58. A pharmaceutical composition comprising Tacrolimus or a close structural analogue thereof wherein the tacrolimus or its close structural analogue is administered not more than once a day at a dose of 0.007 mg/kg to 0.03 mg/kg and wherein the dose is a therapeutically effective amount for treating one or more conditions selected from the group consisting of:
(i) adverse fat accumulation;
(ii) sarcopenia;
(iii) hyperglycaemia or type II diabetes;
(iv) high cholesterol;
(v) high triglycerides;
(vi) metabolic syndrome; and
(vii) combinations thereof.

59. The pharmaceutical composition of claim 58 wherein the dose is not more than 0.1 mg/kg, 0.085 mg/kg or 0.007 mg/kg.

60. The pharmaceutical composition of claim 58 wherein the dose is more than 0.0014 mg/kg or more than 0.002 mg/kg.

61. The pharmaceutical composition of claim 58 wherein the dose is 0.0014 mg/kg to 0.0085 mg/kg, for example 0.002 mg/kg to 0.007 mg/kg.

62. The pharmaceutical composition of claim 58 wherein the dose comprises tacrolimus.

63. A unit dose of a pharmaceutical composition which comprises 0.05 mg to 0.8 mg of tacrolimus or a close structural analogue thereof, wherein the unit dose pharmaceutical composition comprises a therapeutically effective amount for treating one or more conditions selected from the group consisting of:
(i) adverse fat accumulation;
(ii) sarcopenia;
(iii) hyperglycaemia or type II diabetes;
(iv) high cholesterol;
(v) high triglycerides;
(vi) metabolic syndrome; and
(vii) combinations thereof.

64. The unit dose pharmaceutical composition of claim 63 which contains not more than 0.75 mg, 0.6 mg or 0.5 mg.

65. The unit dose pharmaceutical composition of claim 63 which contains not less than 0.05 mg, 0.1 mg or 0.15 mg.

66. The unit dose pharmaceutical composition of claim 63 which contains 0.1 mg to 0.6 mg.

67. The unit dose pharmaceutical composition of claim 63 which contains tacrolimus.

68. The unit dose pharmaceutical composition of claim 63 for oral administration once per day.

69. A unit dose as claimed in claim 63 which is a tablet or capsule.

70. A unit dose of claim 63 which comprises 0.3 mg tacrolimus.

71. A unit dose of claim 63 which comprises 0.5 mg tacrolimus.

72. A unit dose of claim 63 which comprises 0.6 mg tacrolimus.

73. A unit dose of claim 63 wherein the unit dose pharmaceutical composition comprises is a therapeutically effective amount for treating adverse fat accumulation.

74. A unit dose of claim 63 wherein the unit dose pharmaceutical composition comprises is a therapeutically effective amount for treating age related fat accumulation.

75. A method of treatment of one or more conditions in a patient in need thereof which comprises administering not more than once a day an effective amount of tacrolimus or a close structural analogue thereof which amount does not cause immunosuppression and which produces a whole blood trough level of tacrolimus or its close structural analogue of at least 0.05 ng/mL, wherein the one or more condition is selected from the group consisting of:
(i) adverse fat accumulation;
(ii) sarcopenia;
(iii) hyperglycaemia or type II diabetes;
(iv) high cholesterol;
(v) high triglycerides;
(vi) metabolic syndrome; and
(vii) combinations thereof.

76. The method of claim 75 wherein the trough whole blood level produced is at least 0.1 ng/mL.

77. The method of claim 75 wherein the trough whole blood level produced is at least 0.2 ng/mL.

78. The method of claim 75 wherein the trough whole blood level produced is less than 1.2 ng/mL.

79. The method of claim 75 wherein the trough whole blood level produced is less than 1.1 ng/mL.

80. A method of treatment of one or more conditions in a patient in need thereof which comprises administering not more than once a day a dose of tacrolimus or a close structural analogue wherein the dose is from 0.007 mg/kg to 0.03 mg/kg and wherein the one or more conditions is selected from the group consisting of:
(i) adverse fat accumulation;
(ii) sarcopenia;
(iii) hyperglycaemia or type II diabetes;
(iv) high cholesterol;
(v) high triglycerides;
(vi) metabolic syndrome; and
(vii) combinations thereof.

81. A method of claim 80 wherein the dose is not more than 0.1 mg/kg, 0.085 mg/kg or 0.007 mg/kg.

82. A method of claim 80 wherein the dose is more than 0.0014 mg/kg or more than 0.002 mg/kg.

83. A method of claim 80 wherein the dose is 0.0014 mg/kg to 0.0085 mg/kg, or 0.002 mg/kg to 0.007 mg/kg.

84. A method of claim 80 wherein the dose comprises tacrolimus.

85. A method of treatment of one or more conditions in a patient in need thereof which comprises administration not more than once a day of an effective amount of tacrolimus or a close structural analogue sufficient to effect epigenetic modification which leads to a reduction in oxidative stress wherein the one or more conditions is selected from the group consisting of:
(i) adverse fat accumulation;
(ii) sarcopenia;
(iii) hyperglycaemia or type II diabetes;
(iv) high cholesterol;
(v) high triglycerides;
(vi) metabolic syndrome; and
(vii) combinations thereof.

86. A method of reducing serum triglyceride and/or cholesterol which comprises administering to a patient in need thereof 0.05 mg to 0.65 mg of tacrolimus per day.

87. The method of claim 86 which comprises administering to the patient in need thereof 0.1 mg to 0.6 mg of tacrolimus per day.

88. The method of claim 86 which comprises administering to the patient in need thereof 0.2 to 0.45 mg of tacrolimus per day.

89. The method of claim 86 which comprises administering to the patient in need thereof 0.3 mg tacrolimus per day.

90. A method of claim 86 wherein the administration is oral.
